(19) United States
(12) Patent Application Publication
(10) Pub. No.: US 2024/0033393 A1
(43) Pub. Date: Feb. 1, 2024

(54) FAR-UVC EMITTER

(71) Applicant: Alex KING, Las Cruces, NM (US)

(72) Inventor: Alex KING, Las Cruces, NM (US)

(21) Appl. No.: 18/361,620

(22) Filed: Jul. 28, 2023

Related U.S. Application Data
(60) Provisional application No. 63/393,588, filed on Jul. 29, 2022.

(51) Int. Cl. A61L 9/20; F21V 3/00; A61L 2/10; B64D 11/00

(52) U.S. Cl. CPC A61L 9/20; F21V 3/00; A61L 2/10; B64D 11/00; F21W 2107/30

(57) ABSTRACT

A light and sanitization system disclosed herein includes a housing, a ring shaped FAR-UVC emitter configured in the housing, a second light source disposed in the housing and located such that light from the second light source passes through a center of the ring shaped FAR-UVC emitter, and a power source for driving emission of UVC light from the ring shaped FAR-UVC emitter.

### CROSS REFERENCE TO RELATED PATENT APPLICATIONS

The present application claims the priority and benefit under 35 U.S.C. § 119(e) of U.S. Provisional Patent Application Ser. No. 63/393,588 filed Jul. 29, 2022, entitled “FAR-UVC EMITTER.” U.S. Provisional Patent Application Ser. No. 63/393,588 is herein incorporated by reference in its entirety.

### TECHNICAL FIELD

Embodiments are generally related to the field of aviation. Embodiments are further related to the field of lighting. Embodiments are further related to sanitization of enclosed spaces. Embodiments are also directed to methods for sanitizing and lighting enclosed spaces. Embodiments are also related to methods, systems, and devices directed to FAR-UVC emitters for sanitization of such enclosed spaces which can be operated in conjunction with standard lighting assemblies.

### BACKGROUND

The role of air and surface sanitization has become increasingly important given the threat of airborne pathogens. This is acutely true in the transportation industry, where patrons are confined to spaces where sufficient social distancing and other effective sanitization methods may not be possible. It is important to note that airplanes' high-powered filtration systems are not sufficient, on their own, to prevent viral or bacterial transmission. Infected people send particles into the air at a faster rate than airplanes flush them out of the cabin. Passenger aircraft are typically equipped with HEPA filtration which promotes a frequent air exchange rate. However, it is unlikely to protect individuals that are seated in close proximity to an infected passenger.

Current cabin sanitization solutions fall short or create unintended side effects to installed surfaces and/or electrical equipment. The FAA is calling attention to risks that disinfection can have on aircraft interiors, urging operators and maintainers to heed manufacturers' guidance and take extra steps to protect sensitive equipment, wiring, and other high-risk components.

For example, the FAA has noted fogging and misting solutions, especially those which utilize the aircraft or other cabin ventilation system, allow disinfectant to penetrate areas where it could create problems, such as the corrosion of underlying structures or fan-cooled electronics. Likewise, the use of standard germicidal wavelength (254 nm) UV-C equipment is hazardous to human beings and can only be used to sanitize cabins prior to passenger boarding. There are in fact, very limited wavelengths which provide the desired sanitization and are also safe for humans. Therefore, most current options are effective at maintaining a sanitized environment only until the first new passenger boards.

Furthermore, implementation of sanitization technology is particularly difficult on aircraft. First, weight limitations severely restrict the types of systems that can practically be installed on an aircraft. In addition, aircraft safety standards also create specific limitations as to the types of electronic components that can be used on aircraft. Thus, conventional sanitization technology cannot be used on many aircraft.

Scientists have known for decades that UV light has the ability to kill bacteria, and viruses like the common Flu and COVID-19. However conventional UV lamps pose health risks, including damage to skin and eyes. Recent studies by a team of Columbia University researchers show that a narrow-wavelength band of ultraviolet light kills airborne viruses, like COVID-19, without damaging organic tissue like human skin or eyes. Likewise, the use of UVC as a germicidal weapon against viruses, pathogens, mold, spores, etc., is known, but finding a UV light (e.g., wavelength) not harmful to human skin and eyes has remained elusive. Thus, the use of UV light as a mechanism for addressing the need to prevent the spread of disease, viruses, pathogens, mold, and other such sources of human illness is advancing as safe UV exposure levels and wavelengths are identified.

Accordingly, there is a need in the art for methods and systems that address the aforementioned gaps in current technology as disclosed the embodiments detailed herein.

### SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is an aspect of the disclosed embodiments to provide a sanitization system.

It is another aspect of the disclosed embodiments to provide a lighting system.

It is another aspect of the disclosed embodiments to provide light and sanitization system for indoor environments.

It is another aspect of the disclosed embodiments to provide a FAR-UVC emitter capable of continuously and/or periodically providing FAR-UVC light to an environment in order to sanitize the environment. The systems and methods may be configured to operate with, or replace, lighting associated with standard lighting fixtures in environments, including but not limited to airplanes, trains, marine vehicles, busses, cars, buildings, and the like. To that end, the housing for such systems can be configured in a form factor matching, for example, a standard airline passenger service unit (PSU) light.

The aforementioned aspects and other objectives and advantages can now be achieved as described herein. In an exemplary embodiment, a system comprises a FAR-UVC emitter comprising a housing a hex conductor pattern and a metal conductor, a visible light opening in the housing configured to allow light to pass therethrough, and a power source. In an embodiment, the system comprises an opaque coating on the back side of the FAR-UVC emitter configured to prevent upward emission. In an embodiment, the system comprises a diffuser configured in the visible light opening in the housing. In an embodiment, the housing further comprises an outer glass wall configured to contain at least one of a plasma or a gas in the housing. In an embodiment, the system comprises a window formed in the visible light opening in the housing. In an embodiment, the system comprises a second light source. In an embodiment, the second light source comprises a visible light source. In an embodiment, the system comprises a circuit card assembly operably connected to the FAR-UVC emitter configured to drive emission of FAR-UVC light from the FAR-UVC emitter. In an embodiment, the system comprises an in-line power supply configured to provide power to the FAR-UVC emitter.

In another embodiment, a sanitization fixture comprises a housing, a ring shaped FAR-UVC emitter configured so that light can be transmitted through a center of the ring shaped FAR-UVC emitter, and a circuit card assembly configured in the housing. In an embodiment, the circuit card assembly comprises an LED and a power supply. In an embodiment, the circuit card assembly comprises a human eye safe LASER and a power supply. In an embodiment, the sanitization fixture comprises a lens configured at an end of the housing.

In another embodiment an aircraft light and sanitization system comprises a housing, a ring shaped FAR-UVC emitter configured in the housing, a second light source disposed in the housing and located such that light from the second light source passes through a center of the ring shaped FAR-UVC emitter, and a power source for driving emission of UVC light from the ring shaped FAR-UVC emitter. In an embodiment, the aircraft light and sanitization system further comprises a diffuser configured in the opening of the housing. In an embodiment, the aircraft light and sanitization system further comprises a window configured in the opening of the housing. In an embodiment, the second light source comprises a visible light source. In an embodiment, the aircraft light and sanitization system further comprises a circuit card assembly configured with a power supply for driving the second light source. In an embodiment, the housing is configured in a form factor matching a standard airline passenger service unit (PSU) light. In an embodiment, the ring shaped FAR-UVC emitter emits 222 nm FAR-UVC wavelength light.

### BRIEF DESCRIPTION OF THE FIGURES

The accompanying figures, in which like reference numerals refer to identical or functionally similar elements throughout the separate views and which are incorporated in and form a part of the specification, further illustrate the embodiments and, together with the detailed description, serve to explain the embodiments disclosed herein.

FIG. 1A illustrates a perspective view of a FAR-UVC emitter system, in accordance with aspects of the embodiments;

FIG. 1B illustrates an elevation view of a FAR-UVC emitter system, in accordance with aspects of the embodiments;

FIG. 1C illustrates a cross-sectional view of a FAR-UVC emitter system, in accordance with aspects of the embodiments;

FIG. 2A illustrates a light and sanitization system, in accordance with aspects of the embodiments;

FIG. 2B illustrates another light and sanitization system, in accordance with aspects of the embodiments;

FIG. 3A illustrates a drop in light and sanitization system, in accordance with the disclosed embodiments;

FIG. 3B illustrates a cutaway view of a drop in light and sanitization system, in accordance with the disclosed embodiments;

FIG. 4 illustrates an in-line power supply, in accordance with aspects of the disclosed embodiments;

FIG. 5A illustrates a perspective view of a FAR-UVC emitter system, in accordance with aspects of the embodiments;

FIG. 5B illustrates an elevation view of a FAR-UVC emitter system, in accordance with aspects of the embodiments;

FIG. 5C illustrates a cross-sectional view of a FAR-UVC emitter system, in accordance with aspects of the embodiments;

FIG. 6A illustrates a light and sanitization system, in accordance with aspects of the embodiments;

FIG. 6B illustrates another light and sanitization system, in accordance with aspects of the embodiments;

FIG. 7A illustrates a drop in light and sanitization system, in accordance with the disclosed embodiments;

FIG. 7B illustrates a cutaway view of a drop in light and sanitization system, in accordance with the disclosed embodiments;

FIG. 8 illustrates an in-line power supply, in accordance with aspects of the disclosed embodiments;

FIG. 9 illustrates a separated view of a FAR-UVC sanitization system, in accordance with the disclosed embodiments;

FIG. 10 illustrates a separated view of a FAR-UVC sanitization and lighting system, in accordance with the disclosed embodiments;

FIG. 11 illustrates a FAR-UVC sanitization system disposed in an aircraft, in accordance with the disclosed embodiments;

FIG. 12 illustrates a diagram illustrating aspects of a FAR-UVC sanitization system installed in an aircraft, in accordance with the disclosed embodiments; and

FIG. 13 illustrates steps associated with a method for configuring and sanitizing an environment with a FAR-UVC sanitization system, in accordance with the disclosed embodiments.

### DETAILED DESCRIPTION

The particular values and configurations discussed in the following non-limiting examples can be varied, and are cited merely to illustrate one or more embodiments and are not intended to limit the scope thereof.

Example embodiments will now be described more fully hereinafter, with reference to the accompanying drawings, in which illustrative embodiments are shown. The embodiments disclosed herein can be embodied in many different forms and should not be construed as limited to the embodiments set forth herein; rather, these embodiments are provided so that this disclosure will be thorough and complete, and will fully convey the scope of the embodiments to those skilled in the art. Like numbers refer to like elements throughout.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope and concept of the invention as defined by the appended claims.

Various aspects of the disclosed systems and methods are provided herein. In an embodiment, a FAR-UVC “donut” (or ring) shaped emitter is disclosed. The emitter serves as the source that generates a FAR-UVC wavelength (non-visible) light. In certain embodiments, the emitter can comprise an KrCl (Krypton Chloride) micro-plasma flat lamp. It operates using a voltage to excite KrCl gas in the emitter, which creates a plasma and emits a 222 nm FAR-UVC wavelength light. In other embodiments, the emitter can comprise a solid state vacuum sealed nano LED device. The emitter is configured to be installed primarily on aircraft. However, it should be understood, the disclosed systems can be installed on trains, buses, maritime vessels, and other modes of transportation, as well as other environments requiring sanitization and lighting. Other environments could include airports, cinemas, theaters, sporting and concert venues, office spaces, hospitals, and schools.

The emitter can be packaged into a housing and used standalone, or coupled with a light source to deliver lighting, as well as sanitization of air and surfaces continuously, or intermittently, in occupied spaces. The housing can be designed around an existing light's mounting provisions to be mechanical “drop-in” replacements to simplify installation. The “donut” shape of the emitter is designed for aviation applications so that the system can be fit in locations on airplanes where conventional light fixtures and emitters do not fit. The donut shape allows a lamp to provide visible light through the “hole” in the donut, while the emitter provides sanitizing FAR-UVC light.

The system can comprise a power connection, configured to utilize 28 volt DC aircraft power, to power the FAR-UVC emitters. In other embodiments, the power connection can be configured to utilize a 12 volt DC supply, 115/230 volt AC supply, or other such power supply. The emitters can be controlled via simple mechanical means (e.g., turn on/turn off switches), as well as a discrete input (override). They can also be optionally programmable to cycle themselves on and off to regulate dosage levels of the FAR-UVC light.

The emitter is configured to provide a narrow-wavelength band of ultraviolet light which can kill airborne viruses, including but not limited to COVID-19, without damaging organic tissue like human skin or eyes. The system can kill 99.9% of airborne viruses even at very low exposure levels. Exposure levels established by the ACGIH are well above potential exposure levels of the disclosed FAR-UVC light source. Even over long periods of +8 hours, there is no risk of over exposure using the disclosed system continuously. The systems can therefore be used in occupied spaces for disinfecting surfaces and destroying pathogens in the air. The embodiments allow people to be in close proximity to one another without fear of widespread infection.

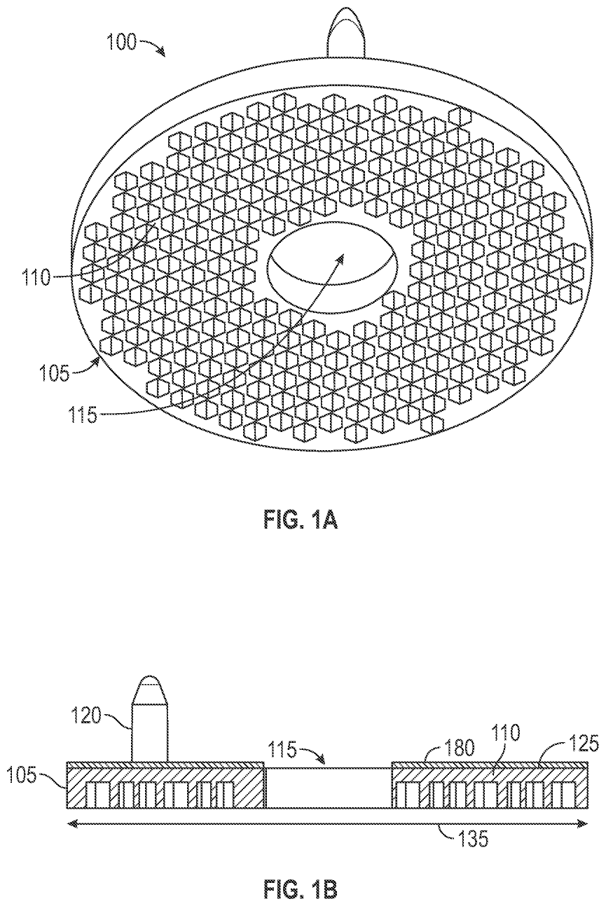

FIG. 1A illustrates aspects of an emitter system 100 in accordance with the disclosed embodiments. The system 100 includes a donut shaped emitter 105, with a hex conductor pattern 110 embedded into the front of the emitter 105. The hex conductor pattern 110 can comprise a gold hex conductor pattern in certain embodiments, but other materials can also be used. A metal (e.g., copper) conductor 180 can also be placed on the backside of the emitter 105. These two conductors are used to create a potential difference, which is used to “excite” the gas inside the emitter 105 to create a plasma.

The emitter 105 can have a ring shape such that there is a tubular ring or opening 115 configured in the center of the emitter 105. The hex conductor pattern is configured in the emitter 105 to facilitate the generation of the FAR-UVC wavelength light which can then pass through the emitter 105. It should be appreciated that the emitter 105 can be comprised of a material (e.g., glass) that allows transmission of FAR-UVC wavelength light.

FIG. 1B provides details of the system 100 in a transparent elevation view, so that internal aspects are visible. As illustrated, the system 100, includes a vacuum port 120 connected to the emitter 105. The emitters 105 are illustrated inside the housing 125 of the emitter 105. The ring 115 is also illustrated in the center of the emitter 105. The system 100 is configured to emit 222 nm FAR-UVC light 135.

FIG. 1C further illustrates additional aspects of the system 100. The emitter system 100 can have an opaque coating 130 configured on the upper facing surface 140 of the housing 125 of the emitter 105, to prevent upward firing in an undesirable direction.

In addition, the ring 115 forming a hole in the center of the emitter 105 can include a diffuser 145 formed in the plane of the downward facing surface 150 of the emitter housing 125. In certain embodiment, the emitter system 100 can be used in conjunction with a light source (LED or Laser based) to emit visible light 155 through the visible light opening 115 or diffuser window 145, to provide diffused visible light 160 as well as FAR-UVC air and surface sanitization from the FAR-UVC light 135.

The internal volume 165 of the emitter 105 can be filled with a gas and/or a plasma 170 (depending on whether the potential difference is being applied). The emitter housing 125 can include an outer wall 175 to contain the plasma 170 or gas. The combination of KrCl (Krypton and Chloride) create the desired 222 nm FAR-UVC wavelength when energized. Neither substance is toxic, making both safe alternatives to conventional UVC lamps which use mercury vapor. In certain embodiments, the outer wall 175 can be glass, but in other embodiments other materials may be used, provided they allow transmission of 22 nm FAR-UVC wavelength light. The plasma/gas 170 in the emitter 105, when supplied with power, results in UVC emission to provide the ultraviolet light 135 for disinfection.

In certain embodiments, the entire housing assembly 125 is comprised of glass, which can be vacuum sealed with Krypton Chloride vapor inside. Two conductors (not shown), which can preferably be honeycomb conductors, can be applied to the emitter, one on the front of the housing and the other on the back of the housing. When voltage is applied to the conductors it excites the gas 170 and creates a plasma that emits 222 nm FAR-UVC light through the emitter 105.

The system 100 can be configured to be installed in association with preexisting ambient lighting. In so doing, the associated environment is provided ambient lighting as well as FAR-UVC sterilizing light.

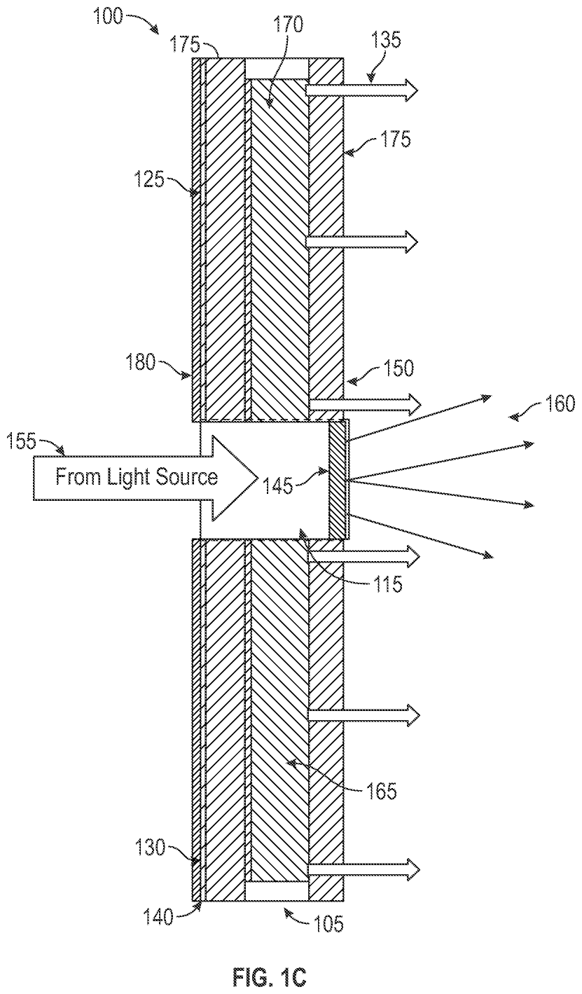

FIGS. 2A and 2B illustrates an exemplary embodiment of the disclosed system 100 installed in association with a fixed light housing 200. The exemplary FAR-UVC enabled fixed light housing 200, as shown in FIG. 2A illustrates an application for custom mechanical drop-in replacement lights as an aspect of the disclosed embodiments. In the exemplary embodiment, the light source 205 can be an LED, or Laser based Surface Mount Diode (SMD), installed as an overhead cabin light in an aircraft ceiling (or other such application). A heat sink 210 can be provided to control the heat load above the fixed light housing 200.

The light source 205 can be installed above a color temperature filter 215. The color temperature filter 215 will provide a “warming” or “cooling” effect to the visible light, and can be designed to support all requested light temperatures. The optic housing 220 can be a tubular housing extending from the light 205, which focuses light generated. A power supply 240 is provided. The donut shaped emitter system 100 can be integrated into the light housing 200, with the upper surface 140 of the emitter 105 mounted up into the lighting fixture.

In certain embodiments, the downward facing side of the ring 115 can be fitted with a beam angle diffuser 145 to distribute the ambient light from the light source 205, at the desired angle 225 to the interior environment 230 (e.g., the aircraft cabin). The beam angle diffuser 145 can optionally be a flood design of approximately 60 degrees, but can also be configured to support any desired angle 225. It should be appreciated that read light designs may be fixed or directional and will typically have a beam angle 225 of approximately 15-20 degrees. Embodiments disclosed herein may be installed in read lights. It should be appreciated that the housing can be custom designed to support the mechanical characteristics of any installation. As such the housing can be any shape (e.g., can be in a form factor matching a standard airline passenger service unit (PSU) light), as long as the design can support the electrical components.

The system 100 installed in connection with a light housing 200 can further include the hex conductor pattern 110 configured to create the potential difference with conductor 180, to provide sanitizing FAR-UVC light to the internal environment 230. The system 100 can also include a decorative bezel 235 configured adjacent to the downward external surface, so that the after-market installation has a pleasing cosmetic appearance.

In certain embodiments, the disclosed systems can include a light housing 200 with an emergency LED circuit, powered by an isolated emergency input. In certain embodiments, the light housing 200 can be configured with no light source such that only the FAR-UVC emitter system 100 provides non-visible sanitizing light, as shown in FIG. 2B.

It should be appreciated that, on aircraft, certain lights are configured to stay powered during an emergency situation to provide some illumination during such an event. Tests are conducted to simulate a smoke filled cabin and test participants will try and crawl to the nearest exit solely with the minimal light given off from a light source while the cabin is “filled with smoke”. Laser based lights sources are preferrable for these types of applications, as they provide a higher “spectral purity”, so they cut through smoke and will yield much more light for emergency situations.

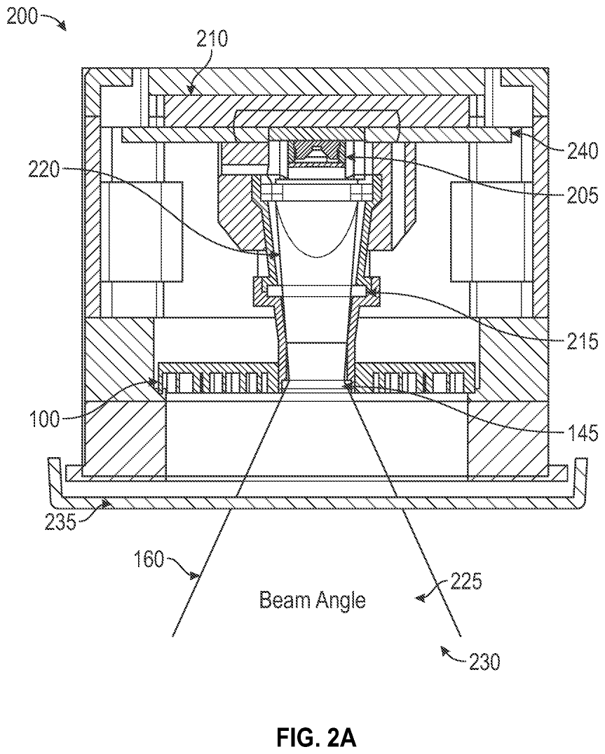

FIGS. 3A and 3B illustrate another embodiment of a system 300 configured to be a mechanical drop-in replacement for a standard airline passenger service unit (PSU) light. FIG. 3A illustrates an elevation view of the system 300, and FIG. 3B illustrates a cutaway view of the system 300.

In such an embodiment, a FAR-UVC emitter 305 can be configured to fit in a PSU light housing 310. The FAR-UVC emitter 305 can be comprised of a donut shaped emitter housing 315 sized to fit in the light housing 310 between the light lens 320 and the reading light 325 and power supply circuit card assembly (CCA) 330. The hole 335 in the FAR-UVC emitter 305 allows light from the light source 325 (e.g., LED or Laser based light source) on the read light power supply CCA 330 to pass through the ring/hole 335 and out the lens 320 of the PSU housing 310.

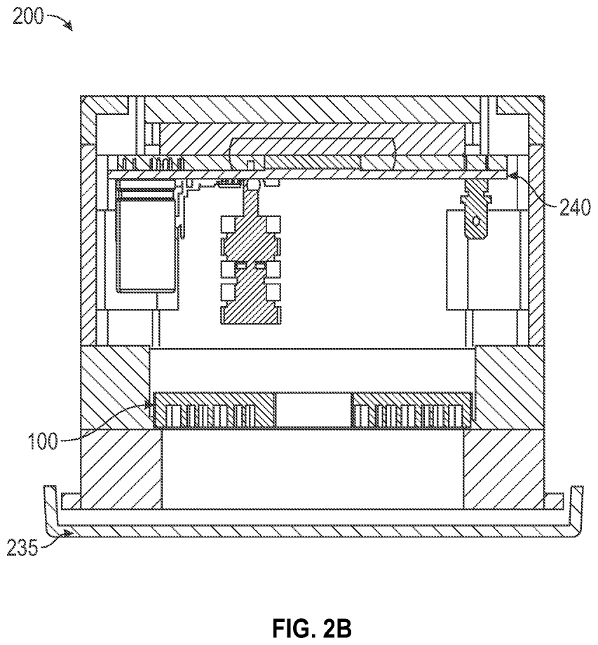

FIG. 4 illustrates an in-line power supply 400. In any of the embodiments disclosed herein, a power supply such as power supply 400 can be provided. The power supply can comprise a custom in-line power supply. In other embodiments, all power supply aspects can be built into the light housing design. The in-line power supply illustrated in FIG. 4, specifically designed for aviation, can accept input aircraft power and supply power to the FAR-UVC enabled devices.

The in-line power supply 400 can take aircraft power and convert it to power that is needed to drive one (or more) FAR-UVC enabled lights. In the PSU design illustrated in FIGS. 3A and 3B, space is extremely limited. As such, an in-line power supply may be required since there is no additional room in the light housing itself. In most other designs, all electronic subcomponents can be built into the light housing.

The power supply 400 can be used to power the emitter. In certain embodiments, the emitter can be run continuously or can be programmed to “pulse” to control the overall dosage level. Running the system continuously (when possible) may be preferrable to maximize the benefit of reducing the viral load of contagions within an enclosed space. The embodiments disclosed herein, can include a mechanical based on/off switch that will allow manual control, to turn the UVC light on or off. The disclosed embodiments can also include an auxiliary discrete input to the light, that will act as an override that can be enabled by, for example, the flight crew as necessary.

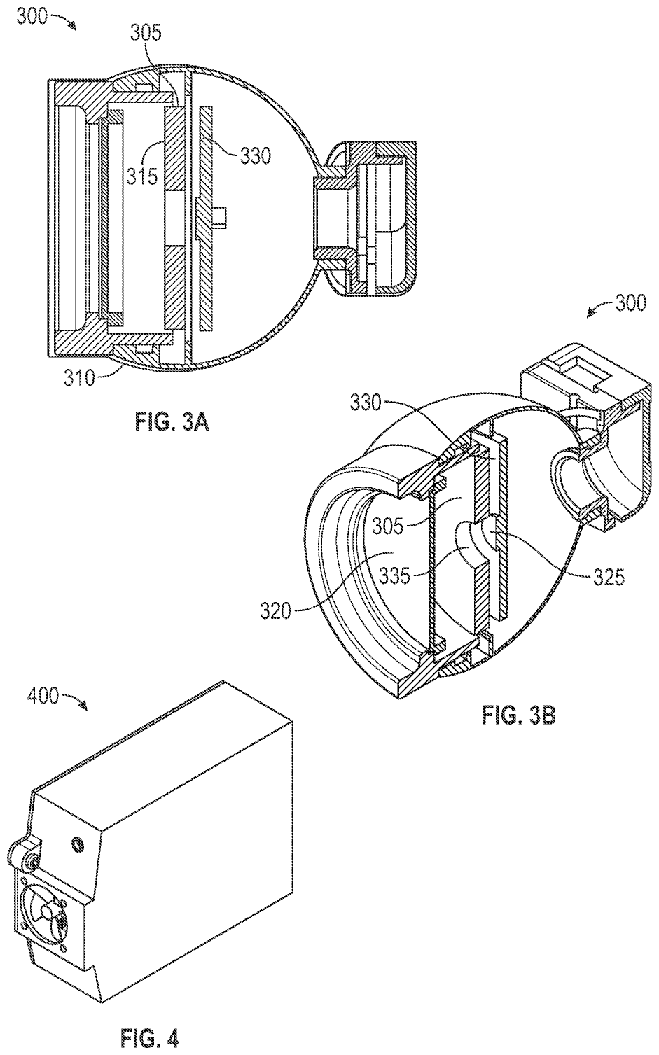

FIGS. 5A-5C illustrate aspects of another embodiment of a system 500. In most respects, the system 500 mirrors that of FIGS. 1-4. As illustrated in FIG. 5A, the system 500 includes a donut shaped emitter 505 with a clear window 545 configured in the center ring 515 of the housing 525. A hex conductor pattern 510 is formed in the emitter 505.

FIG. 5B provides details of the system 500 in a transparent elevation view, so that internal aspects are visible. As illustrated, the system 500, includes a vacuum port 520 connected to the emitter 505. The FAR-UVC hex conductor pattern 510 is illustrated inside the emitter 505 in the housing 525. The hex conductor pattern 510 can comprise a gold hex conductor pattern in certain embodiments, but other materials can also be used. A metal (e.g., copper) conductor 580 can also be placed on the backside of the emitter 505. These two conductors are used to create a potential difference, which is used to “excite” the gas inside the emitter 505 to create a plasma. The ring 515 is also illustrated in the center of the emitter 505. The system 500 is configured to emit 222 nm FAR-UVC light 535.

FIG. 5C further illustrates additional aspects of the system 500. The emitter system 500 can have an opaque coating 530 configured on the upper facing surface 540 of the emitter 505 to prevent upward firing.

In addition, the clear window 545 in the center of the emitter 505 can optionally include a diffuser 546 formed in the plane of the upper facing surface 540 of the emitter 505. In certain embodiments, the emitter system 500 can be used in conjunction with a light source 555 (LED or Laser based) that emits visible light through the diffuser 546 into the clear window 545 to provide ambient light 560 as well as FAR-UVC air and surface sanitization form the FAR-UVC light 535.

The internal volume 565 of the emitter 505 can be filled with a plasma or gas. The emitter housing 525 can include an outer wall 575 to contain the plasma 570 or gas. In certain embodiments, this outer wall 575 can be glass, but in other embodiments other materials may be used. The plasma/gas 570 in the emitter 505, when supplied with power, is used generate the necessary downward UVC emission to provide the ultraviolet light 535 for disinfection.

In another embodiment, a solid state emitter design, which doesn't use Krypton Chloride (KrCl) gas to generate 222 nm wavelength can be used. Instead, the solid-state embodiment uses a vacuum sealed nano LED device for sanitization.

As illustrated herein, the system 500 can be configured to be installed in association with preexisting ambient lighting. The system 500 can be used to provide the associated environment ambient lighting as well as FAR-UVC sterilizing light.

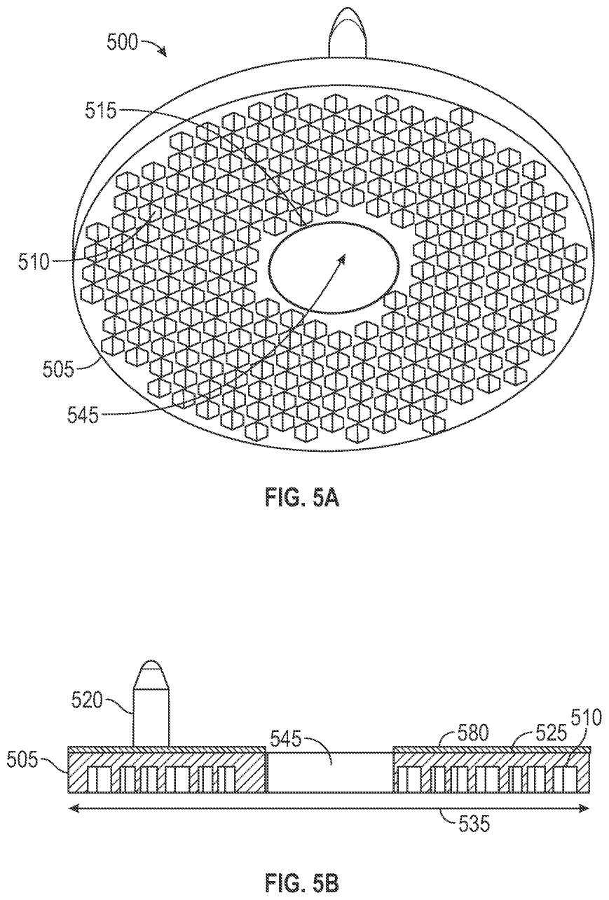

FIGS. 6A and 6B illustrate an exemplary embodiment of the disclosed system 500 installed in association with a fixed light housing 600. The exemplary FAR-UVC enabled fixed light housing 600, as shown in FIG. 6A illustrates an application of a custom mechanical drop-in replacement light, as an aspect of the disclosed embodiments. In this exemplary embodiment, the light source 605 can be an LED, or Laser based SMD, installed as an overhead cabin light in an aircraft (or other such application). A heat sink 610 can be provided to control the heat load above the fixed light housing 600.

The light source 605 can be installed above a color temperature filter 615. The color temperature filter 615 will provide a “warming” or “cooling” effect to the visible light, and can be designed to support all requested light temperatures. The optic housing 620 can be comprised of a tubular housing extending from the light 605, which is configured to focus the light generated by the light source 605. A power supply 640 is provided. The donut shaped emitter system 500 can be integrated into the light housing 600, with the upper surface of the emitter 505 mounted up into the lighting fixture.

The clear window 545 in the emitter 505 can be configured to attach to a beam angle diffuser 546 to diffuse light at the desired angle 625 to the interior environment 630 (e.g., the aircraft cabin). The beam angle diffuser 546 can optionally be a flood design of approximately 60 degrees, but can be designed to support any requested angle 625. In other embodiments, the system can be installed in read light designs which may be fixed or directional ,and will typically have a beam angle 625 of approximately 15-20 degrees.

The system can further include a hex conductor pattern and a metal (e.g., copper) conductor configured to create a potential difference to generate sanitizing FAR-UVC light to the internal environment. The downward external surface can include a decorative bezel 635 so that the after-market installation has a pleasing cosmetic appearance.

In certain embodiments, the disclosed systems can include a light housing 600 with an emergency LED circuit, powered by an isolated emergency input. In certain embodiments, the housing 600 can be configured with no light source such that only the FAR-UVC emitter system 500 provides non-visible sanitizing light, as shown in FIG. 6B.

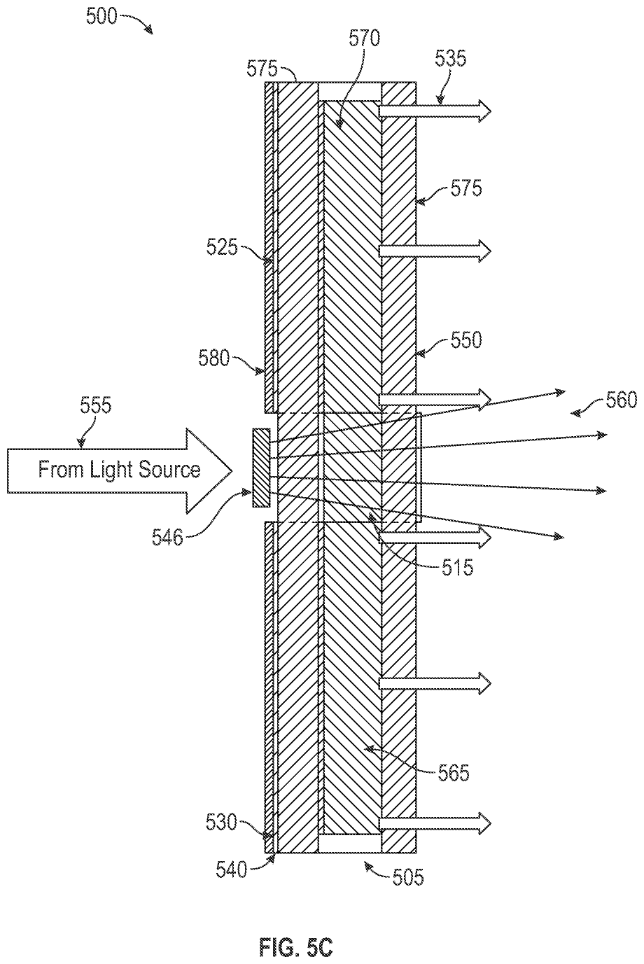

FIGS. 7A and 7B illustrate another embodiment of a system 700 configured to be a mechanical drop-in replacement for a standard airline passenger service unit (PSU) light. FIG. 7A illustrates an elevation view of the system 700, and FIG. 7B illustrates a cutaway view of the system 700.

In such an embodiment, a FAR-UVC emitter 705 can be configured to fit in a PSU light housing 710. The FAR-UVC emitter 705 can be comprised of a donut shaped emitter housing 715 sized to fit in the light housing 710 between the light lens 720 and the reading light 725 and power supply circuit card assembly (CCA) 730.

The clear window 745 in the FAR-UVC emitter 705 allows light from the light source 725 (e.g., LED or Laser based light source) on the read light power supply CCA 730 to pass through the ring/hole 735 and out the lens 720 of the PSU housing 710.

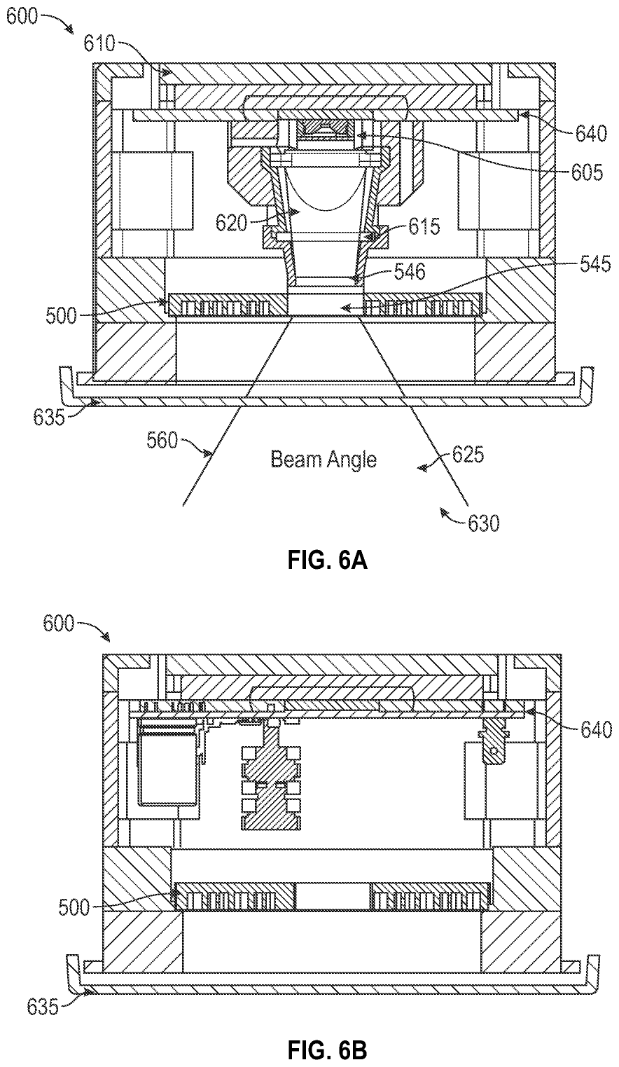

FIG. 8 illustrates an in-line power supply 800. In any of the embodiments disclosed herein, a power supply can be provided. In certain embodiments, the power supply can comprise a custom in-line power supply 800. In other embodiments, all power supply aspects can be built into the light housing design. The in-line power supply 800 illustrated in FIG. 8, is specifically designed for aviation, and is configured to input aircraft power and supply power to the FAR-UVC enabled devices, such as, for example, those illustrated in FIGS. 5-7.

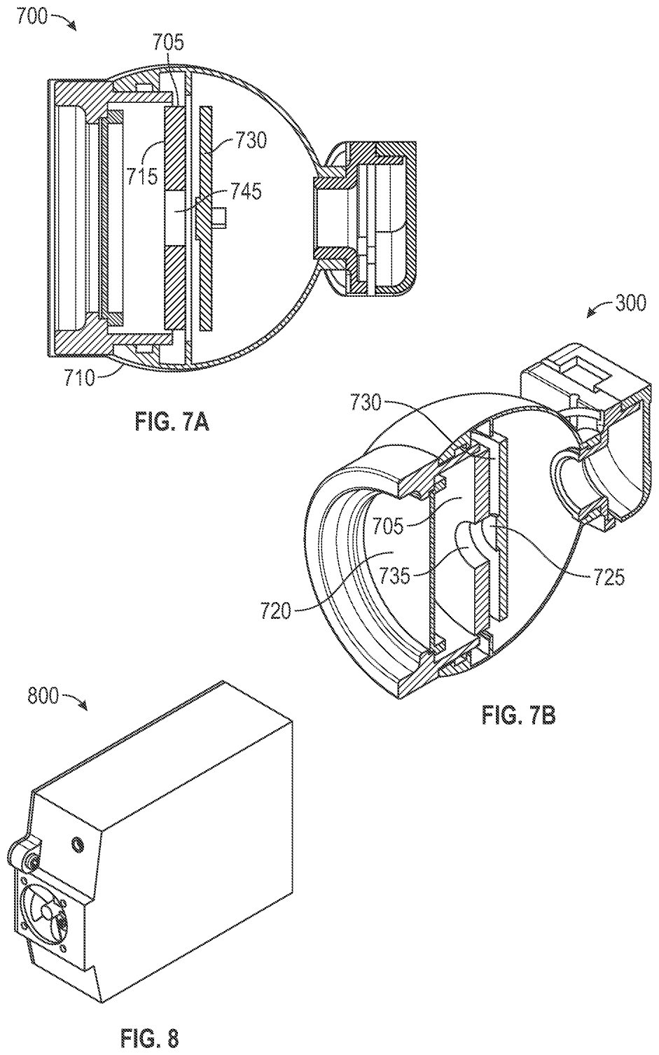

FIG. 9 illustrates another embodiment of a system 900 for providing disinfecting ultraviolet light. In this embodiment, the emitter is configured with a vacuum sealed Nano-LED technology to provide the FAR-UVC light.

The system 900 includes a light housing 905 which can include a tubular housing 910 configured to house a nano FAR-UVC LED emitter 915. A bracket 920 can be used to connect the emitter 915 to the light housing 905. A decorative bezel 925 can be configured to interface between the tubular housing 910 and mounting surface.

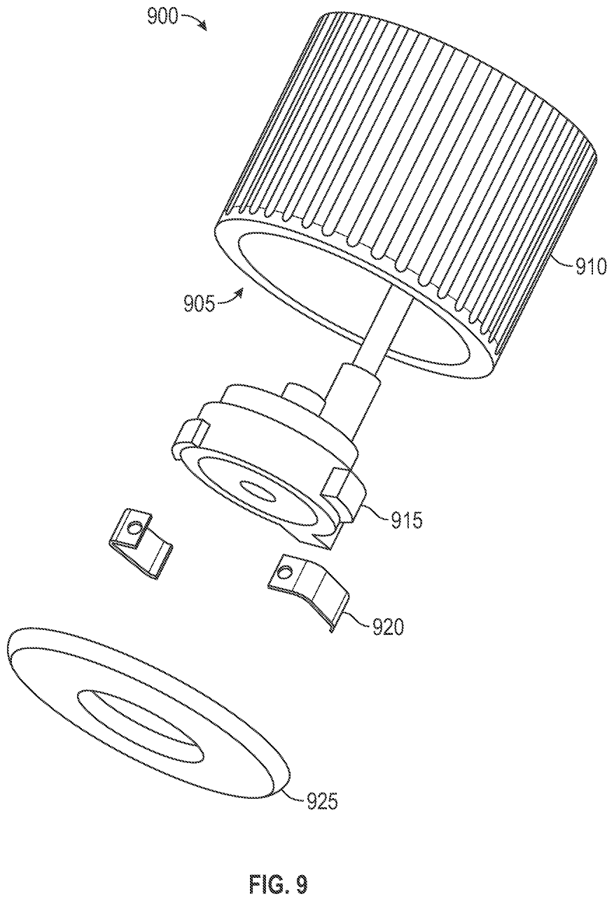

FIG. 10 illustrates another embodiment of a system 1000, configured to provide ambient light as well as disinfecting ultraviolet light. The system 1000 is configured with Nano-LED technology to provide the FAR-UVC light.

In this embodiment, a light housing 1005 can comprise a tubular housing 1010 configured to house a nano FAR-UVC LED emitter 1015. A bracket 1020 can be used to connect the emitter to the tubular housing 1010.

The system further includes a light source 1025 which can comprise an LED or Laser based SMD light source. A parabolic diffuser 1030 and lens 1035 are configured to fit in the housing 1005 creating an optical path in the system 1000. A decorative bezel 1040 can be configured to interface between the tubular housing 1010 and mounting surface. This embodiment can be configured to include an emergency LED circuit, powered by an isolated emergency input.

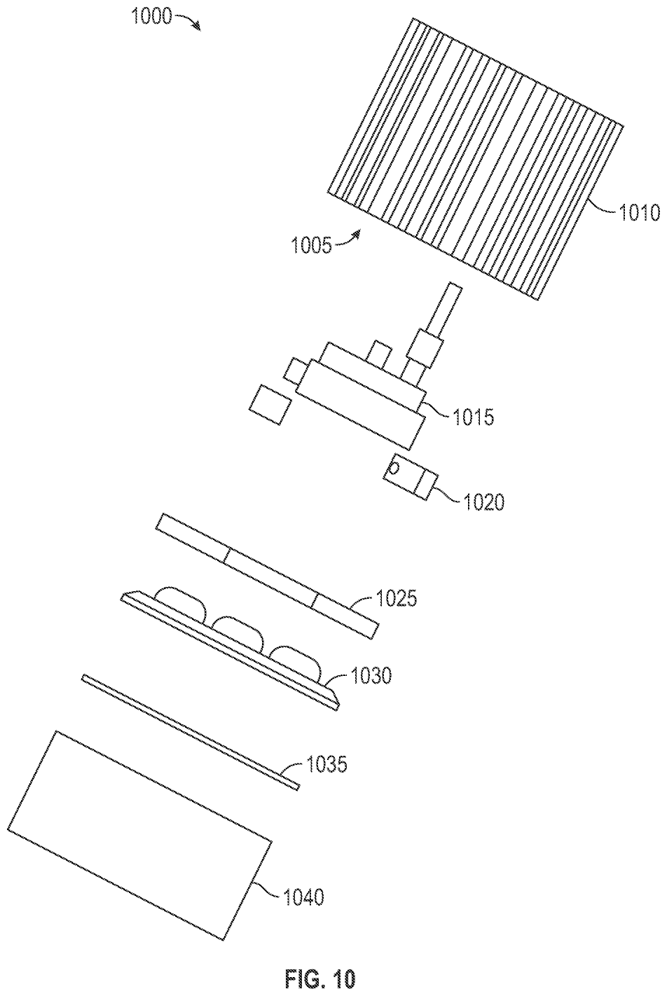

FIG. 11 illustrates an exemplary system 1100 for lighting and sanitizing an aircraft cabin 1105 in accordance with the disclosed embodiments. It should be appreciated that this embodiment is provided to be illustrative, and similar systems can be provided in other enclosed environments without departing from the scope of this disclosure.

As illustrated in FIG. 11, a series of FAR-UVC enabled emitters 1110, which can comprise one or more of the systems, or aspects of the systems illustrated in FIGS. 1-10, can be installed at various locations throughout the cabin 1105 of an aircraft 1115, or other such environment. The system 1100 is configured to provide FAR-UVC light 1120 via FAR-UVC emitters. In certain embodiments, the FAR-UVC light 1120 can comprise 222 nm FAR-UVC light. Optionally, the FAR-UVC emitters 1110 can be installed in the aircraft in conjunction with existing light fixtures 1125.

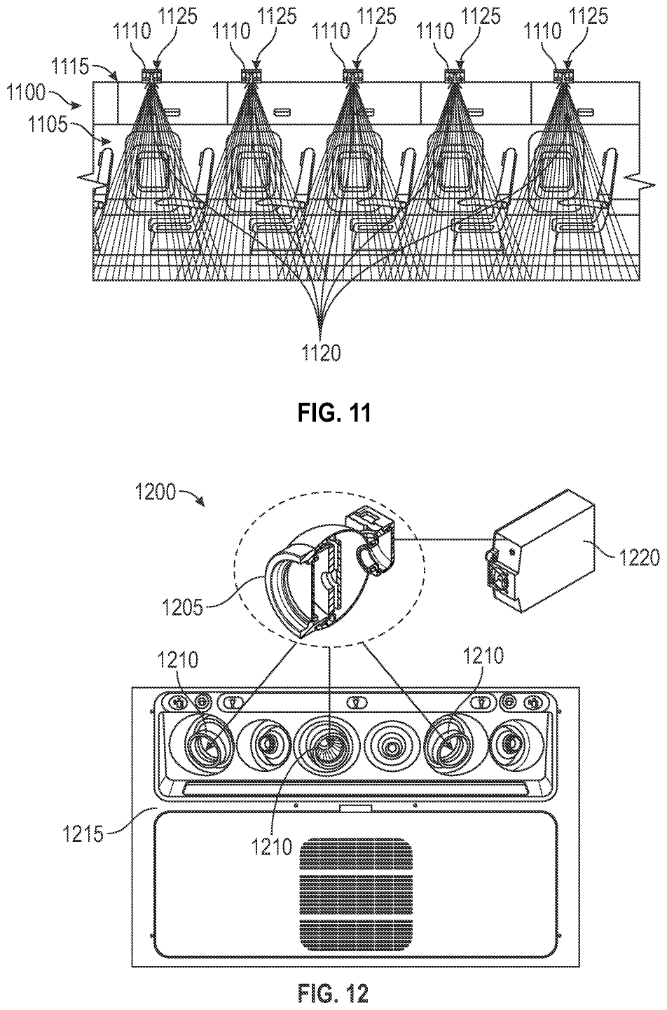

FIG. 12 illustrates another embodiment of a system 1200, wherein a mechanical drop-in PSU light fixtures 1205, which can comprise drop in PSU fixtures as illustrated in FIGS. 3A-3B and 7A-7B, which can be installed in existing PSU light fixture slots 1210 in an aircraft 1215. In this embodiment, a PSU drop in light fixture 1205 (as illustrated in FIGS. 3A-3B and 7A-7B) can be used to replace some or all existing PSU lights, in order to provide lighting and disinfection via FAR-UVC. The light fixtures 1205 can be driven internally, or by an in-line power supply 1220, which can comprise a power supply as illustrated in FIGS. 4 and 8.

Some or all of the hardware associated with the emitters disclosed herein can be designed to pass DO-160 certification guidelines for airworthiness. Table 1 below outlines how all components can meet or exceed DO-160 certification criteria:

TABLE 1

| SECTION | TITLE | CATEGORY |
|---|---|---|
| 4 | Temperature and Altitude | A1 |
| 5 | Temperature Variation | B |
| 6 | Humidity | A |
| 7 | Operational Shocks and Crash Safety | B |
| 8 | Vibration | S |
| | | (CURVE C) |
| 9 | Explosion Proofness | |
| 10 | Waterproofness | Y |
| 11 | Fluids Susceptibility | |
| 12 | Sand and Dust | |
| 13 | Fungus Resistance | F |
| 14 | Salt Spray | |
| 15 | Magnetic Effect | B |
| 16 | Power Input | A, A |
| | | (WF) |
| 17 | Voltage Spike | A |
| 18 | Audio Frequency Conducted Susceptibility - | R, R |
| | Power Inputs | (WF) |
| 19 | Induced Signal Susceptibility | ZC, ZW |
| 20 | Radio Frequency Susceptibility (Radiated | T |
| | and Conducted) | |
| 21 | Emission of Radio Frequency Energy | M |
| 22 | Lightning Induced Transient Susceptibility | BOEING |
| 23 | Lightning Direct Effects | |
| 24 | Icing | |
| 25 | Electrostatic Discharge | A |
| 26 | Fire, Flammability | C |

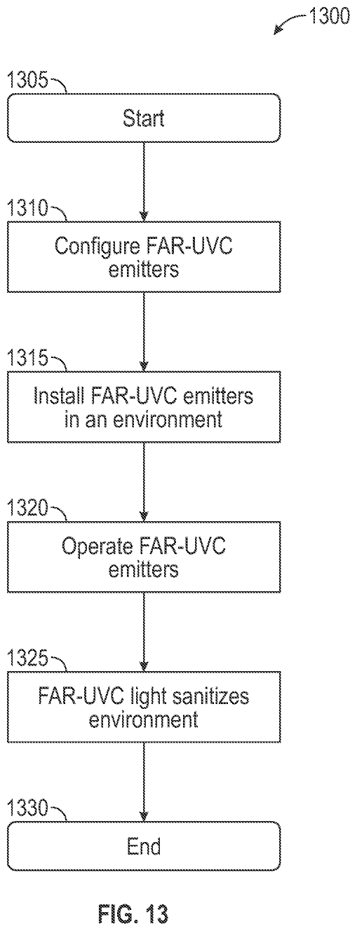

FIG. 13 illustrates a method 1300 for disinfecting an indoor, occupied, or unoccupied, environment in accordance with the disclosed embodiments. The method starts at 1305.

At step 1310, a FAR-UVC emitter as illustrated herein can be fabricated. The emitter can be configured to fit in an existing indoor environment, including but not limited to, an aircraft cabin. The FAR-UVC emitters can be installed in the environment at step 1315. In certain embodiments, the emitters can be installed in, or in association with, existing lighting fixtures.

Once the FAR-UVC emitters are installed, they can be operated at step 1320. This can include using a power supply, which can comprise an in-line power supply as detailed herein. The FAR-UVC emitters can sanitize the associated environment at step 1325, at which point the method ends at 1330.

The disclosed embodiments take advantage of a “narrow-band” of UVC (e.g., 222 nm), coined FAR-UVC, that is an efficient germicidal weapon, but also completely safe for use around humans. In fact, recent studies have shown that long term continuous exposure (30,000 hours) is the equivalent of 10 mins of sunshine in terms of potential skin damage.

As such, some of the disclosed embodiments are custom, simple to install, mechanical drop-in replacement fixtures designed to utilize existing mounting provisions to minimize aircraft integration efforts and downtime. In certain embodiments, the disclosed systems are specifically designed for aviation (DO-160 certified), capable of being installed in small or pre-existing lighting locations. These locations can include galleys, lavatories, walkways, and passenger service units (PSUs).

The disclosed embodiments thus provide FAR-UVC enabled lights including, for example, PSU read lights, which can instantly and continuously sanitize the surrounding air and surfaces.

Based on the foregoing, it can be appreciated that a number of embodiments, preferred and alternative, are disclosed herein. In an embodiment, a system comprises a FAR-UVC emitter comprising a housing a hex conductor pattern and metal conductor, a visible light opening in the housing configured to allow light to pass therethrough, and a power source.

In an embodiment, the system comprises an opaque coating on the back side of the FAR-UVC emitter configured to prevent upward emission. In an embodiment, the system comprises a diffuser configured in the visible light opening in the housing.

In an embodiment, the housing comprises an outer glass wall configured to contain at least one of a plasma or a gas in the housing.

In an embodiment, the system comprises a window formed in the visible light opening in the housing.

In an embodiment, the system comprises a second light source. In an embodiment the second light source comprises a visible light source.

In an embodiment, the system comprises a circuit card assembly operably connected to the FAR-UVC emitter configured to drive emission of FAR-UVC light from the FAR-UVC emitter. In an embodiment, the system comprises an in-line power supply configured to provide power to the FAR-UVC emitter.

In an embodiment a sanitization fixture comprises a housing, a ring shaped FAR-UVC emitter configured so that light can be transmitted through a center of the ring shaped FAR-UVC emitter, and a circuit card assembly configured in the housing. In an embodiment the circuit card assembly comprises an LED and a power supply. In an embodiment the circuit card assembly comprises a human eye safe LASER and a power supply. In an embodiment a sanitization fixture comprises a lens configured at an end of the housing.

In another embodiment an aircraft light and sanitization system comprises a housing, a ring shaped FAR-UVC emitter configured in the housing, a second light source disposed in the housing and located such that light from the second light source passes through a center of the ring shaped FAR-UVC emitter, and a power source for driving emission of UVC light from the ring shaped FAR-UVC emitter.

In an embodiment, the aircraft light and sanitization system comprises a diffuser configured in the opening of the housing.

In an embodiment, the aircraft light and sanitization system comprises a window configured in the opening of the housing.

In an embodiment the second light source comprises a visible light source.

In an embodiment, the aircraft light and sanitization system comprises a circuit card assembly configured with a power supply for driving the second light source.

In an embodiment, the housing is configured in a form factor matching a standard airline passenger service unit (PSU) light.

In an embodiment, the ring shaped FAR-UVC emitter emits 222 nm FAR-UVC wavelength light.

It should be appreciated that variations of the above-disclosed and other features and functions, or alternatives thereof, may be desirably combined into many other different systems or applications. It should be understood that various presently unforeseen or unanticipated alternatives, modifications, variations, or improvements therein may be subsequently made by those skilled in the art which are also intended to be encompassed by the following claims.

What is claimed is:

1. A system comprising:
a FAR-UVC emitter comprising a housing with a hex conductor pattern and a metal conductor;
a visible light opening in the housing configured to allow light to pass therethrough; and
a power source.

2. The system of claim 1 further comprising:
a diffuser configured in the visible light opening in the housing.

3. The system of claim 1 wherein the housing further comprises:
an outer glass wall configured to contain at least one of a plasma or a gas in the housing.

4. The system of claim 3 further comprising:
an opaque coating on the back side of the FAR-UVC emitter configured to prevent upward emission.

5. The system of claim 1 further comprising:
a window formed in the visible light opening in the housing.

6. The system of claim 1 further comprising:
a second light source.

7. The system of claim 6 wherein the second light source comprises a visible light source.

8. The system of claim 1 further comprising:
a circuit card assembly operably connected to the FAR-UVC emitter configured to drive emission of FAR-UVC light from the FAR-UVC emitter.

9. The system of claim 1 further comprising:
an in-line power supply configured to provide power to the FAR-UVC emitter.

10. A sanitization fixture comprising:
a housing;
a ring shaped FAR-UVC emitter configured so that light can be transmitted through a center of the ring shaped FAR-UVC emitter; and
a circuit card assembly configured in the housing.

11. The sanitization fixture of claim 10 wherein the circuit card assembly comprises:
an LED; and
a power supply.

12. The sanitization fixture of claim 10 wherein the circuit card assembly comprises:
a human eye safe LASER; and
a power supply.

13. The sanitization fixture of claim 10 further comprising:
a lens configured at an end of the housing.

14. An aircraft light and sanitization system comprising:
a housing;
a ring shaped FAR-UVC emitter configured in the housing;
a second light source disposed in the housing and located such that light from the second light source passes through a center of the ring shaped FAR-UVC emitter; and
a power source for driving emission of UVC light from the ring shaped FAR-UVC emitter. cm 15. The aircraft light and sanitization system of claim 14 further comprising:
a diffuser configured in the opening of the housing.

16. The aircraft light and sanitization system of claim 14 further comprising:
a window configured in the opening of the housing.

17. The aircraft light and sanitization system of claim 14 wherein the second light source comprises a visible light source.

18. The aircraft light and sanitization system of claim 17 further comprising:
a circuit card assembly configured with a power supply for driving the second light source.

19. The aircraft light and sanitization system of claim 14 wherein the housing is configured in a form factor matching a standard airline passenger service unit (PSU) light.

20. The aircraft light and sanitization system of claim 14 wherein the ring shaped FAR-UVC emitter emits 222 nm FAR-UVC wavelength light.
